(19) United States
(12) Patent Application Publication
(10) Pub. No.: US 2011/0045593 A1
(43) Pub. Date: Feb. 24, 2011

(54) Transgenically mitigating the establishment and spread of transgenic algae in natural ecosystems by suppressing the activity of carbonic anhydrase

(75) Inventors: Ofra Chen, Rehovot (IL); Shai Ufaz, Givat-Ada (IL); Doron Eisenstadt, Haifa (IL); Daniella Schatz, Givataim (IL); Jonathan Gressel, Rehovot (IL); Shai Einbinder, Hofit (IL)

Correspondence Address: DODDS & ASSOCIATES, 1707 N STREET NW, WASHINGTON, DC, 20036, US

(73) Assignee: TransAlgae (Israel) Ltd., Rehovot (IL)

(21) Appl. No.: 12/806,613

(22) Filed: Aug. 17, 2010

Related U.S. Application Data
(60) Provisional application No. 61/274,608, filed on Aug. 19, 2009.

(51) Int. Cl. C12N 15/82; C12N 15/74

(52) U.S. Cl. 435/471

(57) ABSTRACT

Genetic mechanisms for mitigating the effects of introgression of a genetically engineered genetic trait of cultivated algae or cyanobacteria to its wild type or to an undesirable, interbreeding related species, as well as preventing the establishment of the transgenic algae or cyanobacteria in natural ecosystems by suppressing the activity of the carbon concentrating mechanism.

### PRIORITY

This application claims priority of the U.S. Provisional application No. 61/274,608 filed on Aug. 19^{th} 2009.

### SEQUENCE LISTING

This application contains a sequence listing which is provided in paper format and on computer readable diskette.

### FIELD OF THE INVENTION

The present invention relates to a genetic mechanism for preventing the establishment of transgenic algae and cyanobacteria in natural ecosystems should they be released from enclosed cultivation.

### BACKGROUND OF THE INVENTION

Algae and cyanobacteria have recently attracted much interest as biofactories for production of foods, bioactive compounds and biofuels. Since algae and cyanobacteria need sunlight, carbon-dioxide, and water for growth, they can be cultivated in open or enclosed water bodies. These systems are vulnerable to being contaminated by other algal species and cyanobacteria. Similarly, the cultivated algae may escape outside the cultivation system. This may become a serious concern when the cultivated cells are transgenically modified.

The release of organisms containing introgressed genetically engineered genetic traits may have negative environmental impacts and be of regulatory concern, and thus it is imperative that algae and cyanobacteria containing transgenic traits do not establish outside of their place of cultivation. While the major type of introgression from transgenic crops is sexual interspecific genetic gene flow, and in some cases sexual gene flow to related species, in the case of algae and cyanobacteria it is mainly that they themselves will establish and propagate asexually, as sexual exchanges are quite rare with most algal and cyanobacterial species. Still, cyanobacteria can be subject to horizontal gene flow through phages and possibly by conjugation. Horizontal gene flow is rare in eukaryotic organisms including algae, but conjugation-like processes have been confirmed, intra-specifically in the laboratory by protoplast fusion (Sivan and Arad, 1998). What can occur in the laboratory at high frequency intra-specifically, can happen at much lower frequencies in nature, posing a finite risk, possibly even between related species.

Algae and cyanobacteria have only recently been considered for wide scale cultivation with the process of domestication limited mainly to selection of organisms, occasionally with selection of strains or mutants with desired traits. Unlike with crops, millennia of efforts have not been invested in their domestication, and in many cases the traits needed, do not exist within the species. Genetic engineering allows one to rapidly fill the void of needed traits for rapid domestication (e.g. Gressel, 2008a). Indeed, large scale cultivation of algae has been plagued by problems that are analogous to agricultural production of crops (Gressel 2008b; Sheehan et al., 2004). These problems include contamination by other algae and cyanobacteria (analogous to weeds in crops), fungi, bacteria, viruses (analogous to pathogens of crops), zooplankton (analogous to arthropod pests of crops), low productivity, and especially the lack of certain desired traits (dealt within crops by breeding for millennia). With crops, the analogous comparable problems with cultivation, light penetration, light use efficiency, heating, mineral nutrition, and harvesting have been dealt with by breeding coupled with development of novel cultivation procedures, and continued with the added tools of genetic engineering, which allows introducing traits not available in the genome of the organism.

The needed traits could be artificially introduced into the algae and cyanobacteria by genetic engineering to enhance cost-effectiveness (higher yields, new products, resistances to contaminations, adaptability to cultivation with high levels of light and carbon dioxide not presently occurring in their natural ecosystems). Detractors of both the process of genetic engineering and its products have raised the possibilities that the engineered algae and cyanobacteria would become uncontrollable problems if there was an inadvertent leak or spill from cultivation systems into natural ecosystems. The benefits that accrue from cultivating transgenic algae and cyanobacteria, with their much higher primary productivity than terrestrial crops, could have great benefits to humanity by providing equivalent products on far less land area than conventional agriculture, often using seawater instead of potable fresh water, with far less fertilizer, and without fertilizer or pesticide run-off, allowing removal of agricultural land from production and putting the land to more environmentally sound use.

There is thus a recognized need for, and it would be highly advantageous to have, failsafe anti-establishment, or establishment-mitigating mechanisms to reduce the possibility of establishment of algae and cyanobacteria released to natural ecosystems that will also preclude establishment of rare cases where the transgenes interspecifically introgress into other algae or cyanobacteria.

### SUMMARY OF THE INVENTION

In order to address the drawbacks of current technologies, we here extend our previously described concept for higher plants to algae and cyanobacteria; tandemly combine a gene that is needed in the transgenic algae or cyanobacteria and poses a risk in natural ecosystems, with another gene that is either useful or neutral to the cultivated algae or cyanobacteria, but would be deleterious to the organisms in natural ecosystems such that there is a net fitness disadvantage. Because of the tandem construct, the genes remain genetically linked through asexual or sexual propagation, or gene flow. In cases where there is no sexual or asexual recombination, the genes may be introduced separately.

Thus, a gene that has either a neutral or desirable effect on the algae and cyanobacteria in cultivation in an environment containing high levels of carbon dioxide, but will prevent competition and establishment in the natural environment is genetically engineered into the algae and cyanobacteria in tandem with another gene that might supply a selective advantage to the organism. This would override any selective advantage derived from transgenes that might provide a modicum of advantage in natural ecosystems. In this case we specifically use transgenic constructs that suppress the action of the carbon concentrating mechanism, necessary for life of algae and cyanobacteria in natural ecosystems, but unnecessary in the high carbon dioxide environment of specialized cultivation systems.

According to the present invention a method is provided to obtain transgenic algae or cyanobacteria bearing at least one genetically engineered, commercially desirable genetic trait that is at risk of establishing in natural ecosystems (Table 1), but is tandemly linked to, and co-expressing at least one transgene (mitigating gene) that is desirable in, or neutral to the cultivated transgenic algae or cyanobacteria but rendering the transgenic algae or cyanobacteria incapable of establishing by itself or in introgressed offspring in natural ecosystems. Interfering with the function of the carbon concentrating mechanism will not interfere with cultivation of algae or cyanobacteria in the presence of high levels of carbon dioxide but will preclude their ability to live in natural habitats where the ambient carbon dioxide concentration is too low to allow them to photosynthesize. Thus, any gene that suppresses or inhibits the formation of an intracellular CO₂ pool in the cultivated algae or cyanobacteria will mitigate the effects of release of said genetically engineered, commercially desirable genetic trait of the algae or cyanobacteria by preventing establishment in natural ecosystems. The sequences encoding the desirable genetic traits and the sequences of the mitigating gene remain genetically linked in the transgenic algae or cyanobacteria according to this invention, because of the introduction of the sequences in tandem. If there is no sexual or pseudosexual recombination in the species, the genes need not be tandemly linked.

According to the present invention the transgene that prevents the establishment of the algae or cyanobacteria may be, one or more of the following:

1. A transgene encoding carbonic anhydrase (such as an antisense or RNAi construct of the carbonic anhydrase encoding gene) targeted toward the pyrenoid centers of carbon dioxide fixation within the chloroplasts, or to the carboxysome that allows normal growth of the algae or cyanobacteria only at artificially high carbon dioxide concentrations, but not in natural environments;
2. A transgene encoding production of a carbonic anhydrase inhibitor such as porcine carbonic anhydrase protein inhibitor, GenBank accession number U36916 (Wuebbens et. al 1997) that also only allows normal algae or cyanobacteria growth at artificially high carbon dioxide concentrations, but not in natural environments. In cases where there is no recombination in the species, the gene of choice can be introduced into a mutant strain having a reduced photosystem II antennae.
3. A transgene that encodes the constant production of a cytoplasm carbonic anhydrase (such as Synechococcus PCC 7942, GenBank accession no: M77095)
4. A transgene that encodes the constant production of a chloroplast carbonic anhydrase (such as Arabidopsis, GenBank accession no: NP 568303);
5. The above genes can be together with any other transgene that is neutral or beneficial to the algae or cyanobacteria when cultivated commercially, but renders the algae or cyanobacteria unfit to compete in natural ecosystems, overcoming any benefit that may derive from the transgene tandemly bound to it. Having more than one mitigating gene in the organism supplies an added modicum of biosafety. Such other mitigating genes are summarized in Table 2.

Thus, a method is provided to obtain a cultivated algae or cyanobacteria having multiple transgenes in tandem, (or in some cases separately introduced), derived from different sources with at least one of the transgenes capable of mitigating the fitness effects preventing stable establishment of at least one genetically engineered, commercially desirable genetic trait of the algae or cyanobacteria in natural ecosystems.

According to yet another aspect of the present invention there is provided a method of obtaining cultivated asexual, non-conjugating algae or cyanobacteria capable of mitigating the effects of self propagation of at least one genetically engineered, commercially desirable genetic trait in natural ecosystems. This method comprises transforming a population of the cultivated algae or cyanobacteria to express at least one genetically engineered, commercially desirable genetic trait into algae or cyanobacteria bearing a natural or induced mutation that acts as a mitigating genetic trait, wherein said mitigating genetic trait is selected such that a self propagated said mitigating genetic trait is less fit than native algae or cyanobacteria not expressing said mitigating genetic trait.

According to further features in preferred embodiments of the invention described below, at least one commercially desirable genetic trait is selected from the group consisting of herbicide resistance, resistance to disease and/or zooplankton predation, environmental stress resistance, the ability to fluoresce near-ultraviolet light to photosynthetically usable light, high productivity, modified polysaccharide, protein or lipid qualities and quantities, enhanced yield, expression of heterologous or homologous products and other genetically modified algae and cyanobacteria products.

According to yet further features in preferred embodiments of the invention described below, the gene suppressing the carbon concentrating mechanism can be coupled with one or more other mitigating genetic traits selected from the group consisting of decreased RUBISCO, decreased storage or cell wall polysaccharides, decreased chlorophyll and/or carotene, decrease enzymatic expressions of enzymes that catalyze essential metabolic pathway (such as nitrate reductase, which is not essential when cells are cultured on ammonium, but is essential in nature), decreased or eliminated motility organs, and increased storage materials that cannot be easily catabolized to add a greater degree of biosafety by reducing the risk of establishment outside of specialized cultivation.

According to still further features in preferred embodiments of the invention described below, at least one mitigating genetic trait is a reduced expression of endogenous genetic trait of said cultivated algae or cyanobacteria.

According to further features in preferred embodiments of the invention described below, the cultivated algae or cyanobacteria is one of the following Synechococcus PCC7002, Phaeodactylum tricornutum, Nannochloropsis sp. CS-246, Nannochloropsis oculata, Nannochloropsis salina, Pavlova lutheri CS-182, Synechococcus PCC7942, Synechosystis PCC6803, Chlamydomonas reinhardtii, Chlorella vulgaris, Chlorella spp., Isochrysis sp. CS-177, Tetraselmis chuii CS-26 Tetraselmis suecica CS-187, Nannochloris spp., and the commercially desirable genetic trait is single, double or triple herbicide resistance, and the mitigating genetic trait leads to transformants that have an obligate requirement for high CO₂ concentrations for growth.

According to still further features in preferred embodiments of the invention described below, the first and second polynucleotides (i.e. sequences encoding mitigating and beneficial traits) are integrated separately into an organism that has no known ability to exchange DNA among cells.

According to further features in preferred embodiments of the invention described below, at least one commercially desirable genetic trait is selected from the group consisting of herbicide resistance, disease and/or zooplankton resistance, environmental stress resistance, the ability to fluorescence near-ultraviolet light to photosynthetically usable light, high productivity, modified polysaccharide, protein or lipid qualities and quantities, enhanced yield, and expression of heterologous or homologous products and other genetically modified algae and cyanobacteria products.

The present invention successfully addresses the shortcomings of the presently known configurations by conceiving and providing a mechanism for mitigating the establishment of the transgenic algae or cyanobacteria and their progeny from establishing by self-propagation or by the effects of introgression of a genetically engineered genetic trait of an alga or cyanobacterium to competing organisms. In the case of asexual organisms, where conjugation is unknown, it is sufficient that the mitigating gene be an irreversible mutation to a mitigating form.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIGS. 1A and B Suppression of carbonic anhydrase activity by 300 μM ethoxyzolamide (EZA; 6-ethoxyzolamide; 6-ethoxy-2-benzothiazolesulfonamide) interferes with carbon dioxide uptake and thus decreases photosynthetic carbon dioxide fixation (measured as oxygen evolution with an oxygen electrode) in cultures of Nannochloropsis oculata CS-179 when measured in low CO₂ concentration (1B), while no effect is seen when measured in high CO₂ concentration (1A). Cultures were incubated with ethoxyzolamide just prior to measurement.

FIGS. 2 A and B. Schematic diagram of constructs used to induce the over-expression of the pds (phytoene desaturase) herbicide resistant gene in tandem with the carbonic anhydrase inhibitor gene (pica) in algae pSI103 expression vector (FIG. 2A) and in cyanobacteria pCB4 expression vector (FIG. 2B). The pds and the carbonic anhydrase inhibitor are cloned each in pSI103 under the control of Hsp70-RbcS2 promoters, but other promoters can also be used. In cyanobacteria pCB4 vecoter the genes are cloned under control of RbcLS promoter, but other promoters can also be used.

FIGS. 3A and B. Schematic diagram of constructs used to induce the RNAi of Isochrysis galbana carbonic anhydrase. An inverted repeat of the first 240 bp from the carbonic anhydrase-coding region is cloned downstream to the pds (phytoene desaturase) gene which confers resistance to fluorochloridone (3A). FIG. 3B shows a construct where advantageous transgene is a blue fluorescent protein encoding gene (3B). The transgene in both of these examples is under the control of the Hsp70-RbcS2 promoter and RbcS2 terminator in algae pSI103 expression vector, but other promoters and terminators can be used.

FIG. 4. Schematic diagram of construct used to induce the over-expression of the ppo herbicide resistant gene in tandem with the over-expression of the Arabidopsis chloroplast carbonic anhydrase (AtCA), each controlled by the Hsp70-RbcS2 promoters in algae pSI103 expression vector, but other promoters can be used.

FIG. 5. Schematic diagram of construct used to induce the over-expression of the ppo herbicide resistant gene in tandem with the over expression of the Synechococcus PCC 7942 cytoplasmatic carbonic anhydrase (SynCA), each controlled by the Hsp70-RbcS2 promoters in algae pSI103 expression vector, but other promoters can be used.

### DETAILED DESCRIPTION OF THE INVENTION

The present invention is of genetic mechanisms that can be used for preventing the establishment of transgenic algae or cyanobacteria in natural ecosystems and mitigating the effects of introgression of a genetically engineered genetic trait of a cultivated algae or cyanobacteria to an undesirable, related species of the algae or cyanobacteria. Specifically, the present invention can be used to preclude the establishment of self-propagated transgenic algae or cyanobacteria and mitigating the effects of introgression of genetically engineered traits related algae or cyanobacteria.

The principles and operation of the present invention may be better understood with reference to the accompanying description and examples.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Generally, the nomenclature used herein and the laboratory procedures in recombinant DNA technology described below are those well known and commonly employed in the art. Standard techniques are used for cloning, DNA and RNA isolation, amplification and purification. Generally, enzymatic reactions involving DNA ligase, DNA polymerase, restriction endonucleases and the like, are performed according to the manufacturers' specifications. Generally, the nomenclature used herein and the laboratory procedures utilized in the present invention include molecular, biochemical, microbiological and recombinant DNA techniques. Such techniques are thoroughly explained in the literature. See, for example, Sambrook et al., (1989); Ausubel, R. M., ed. (1994); Ausubel et al (1989); Perbal, (1988); Watson et al., (1998); methodologies as set forth in U.S. Pat. Nos. 4,666,828; 4,683,202; 4,801,531; 5,192,659 and 5,272,057; Cellis, J. E., ed. (1994); Coligan J. E., ed. (1994); Stites et al. (eds.), (1994); Mishell and Shiigi (eds.), (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; Gait, M. J., ed. (1984); Hames, B. D., and Higgins S. J., eds. (1985); Hames, B. D., and Higgins S. J., eds. (1984); “Freshney, R. I., ed. (1986);” (1986); Perbal, B., (1984) and “Methods in Enzymology” Vol. 1-317, Academic Press; “(1990); Marshak et al., (1996). Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

As used herein the terms “genetically linked” and “tandem” refers to a genetic distance smaller than 50 centiMorgan, preferably smaller than 40 centiMorgan, more preferably smaller than 30 centiMorgan, more preferably smaller than 20 centiMorgan, more preferably smaller than 10 centiMorgan, more preferably smaller than 5 centiMorgan, more preferably smaller than 1 centiMorgan, most preferably in the range of 0 to 1 centiMorgan, wherein 0 centiMorgan refers to juxtaposed sequences.

One of the greatest advantages of herbicide-resistant algae and cyanobacteria is that they allow control of closely-related algae and cyanobacteria that have the same herbicide selectivity spectrum as the cultivated algae and cyanobacteria and could not be previously controlled. Similarly, an advantage of disease resistant algae and cyanobacteria is that they will not be decimated by pathogens. Highly productive algae and cyanobacteria are also advantageous, as are algae and cyanobacteria with modified product such as different types of starch and oils. These, and other genetic traits have been, or could be, transgenically introduced into algae or cyanobacteria of various types (see Table 1, herein below).

TABLE 1
Commercially desirable traits that can be engineered into algae and cyanobacteria that
may be undesirable if algae or cyanobacteria are released into natural ecosystems. This list is not
an exclusive list, but one skilled in the art would be able to select other desirable traits.

| Trait | Genetic Element | Source |
|---|---|---|
| Fatty acid | delta(12)-fatty acid dehydrogenase | various |
| composition | (fad2), | |
| Fatty acid | fatty acid desaturase | various |
| composition | | |
| Fatty acid | thioesterase (TE) | Umbellularia californica |
| composition | | |
| Enhanced | Aldolase and TPI D-fructose 1,6- | |
| photosynthesis | bisphosphatase/sedoheptulose 1,7- | |
| | bisphosphatase | |
| Enhanced | Blue Fluorescent protein | |
| photosynthesis | | |
| Increased | Overexpressed cystathione γ-synthase | Heterologous plant or bacterial |
| methionine content | | |
| Increased lysine | Elevated dihydropicolinate synthase | Mutant bacteria |
| content | and | Endogenous antisense or RNAi |
| | suppressed lysine ketobutyrate | |
| | reductase/saccharopine dehydrogenase | |
| Herbicide resistance | 5-enolpyruvylshikimate-3-phosphate | Agrobacterium tumefaciens CP4 or |
| | synthase (EPSPS) | Zea mays mutants |
| Herbicide resistance | Phytoene desaturase | Hydrilla |
| Herbicide resistance | glyphosate oxidoreductase | Ochrobactrum anthropi |
| Herbicide resistance | acetolactate synthase | Various sources |
| Herbicide resistance | Nitrilase | Klebsiella pneumoniae subspecies |
| | | ozanae |
| Herbicide resistance | phosphinothricin N-acetyltransferase | S. hygroscopicus or S. viridochromogenes |
| Herbicide resistance | 4-hydroxyphenyl-pyruvate-dioxygenase | Arabidopsis |
| | (HPPD) | |
| Herbicide resistance | Protoporphyrinogen oxidase | Amaranthus tuberculantus |
| | (PPO or protox) | |
| Herbicide | Glutamine synthetase | Rice, pea, others |
| resistance& | | |
| Increase total amino | | |
| acid and biomass | | |
| contents | | |
| Mercury | merA + merB | Mercury resistant bacteria |
| volatilization | | |
| Virus/phage | Helicase | From pathogens |
| resistance | | |
| Virus/phage | replicase | From pathogens |
| resistance | | |
| Virus resistance | viral coat protein | From pathogens |
| Anti microbia/ | Clavanin A | tunicate |
| fungal peptide | | |
| Anti microbial/ | Penaeidin | shrimp |
| fungal peptide | | |
| Anti microbial/ | Tachypelsin | horseshoe crab |
| fungal peptide | | |
| Anti microbial/viral | Tilapia hepcidin 1-5 (TH 1-5) | tilapia |
| peptide | | |
| Anti microbial/viral | (cSALF) | shrimp |
| peptide | | |
| Anti microbial/ | pleurocidin | flounder |
| fungal peptide | | |
| Anti microbial/ | Magainin2 | Frog |
| fungal | | |
| protozoa | | |
| Anti microbial/ | Phylloseptins | Frog |
| fungal | | |
| protozoa | | |

The advantages of transgenics are well appreciated, if there is no danger of establishment of the transgenic algae or cyanobacteria in natural ecosystems or introgression into a related alga or cyanobacterium. Because the advantages of transgenics are so great, as in the above cases, new, modified transgenic algae and cyanobacteria are being developed.

Hence, while conceiving the present invention, the concept of mitigating the risks of establishment in natural ecosystems or introgression of a genetically engineered trait from the cultivated algae or cyanobacteria, it was conceived that the primary gene of choice having the desired trait (Table 1) should be in tandem constructs with a gene suppressing the synthesis or activity of carbonic anhydrase. This latter gene can be coupled with other “anti-establishment”, mitigating genes (Table 2) also conferring a disadvantage on the algae, or cyanobacteria, or into introgressed progeny when in natural ecosystems, while being benign or advantageous to the cultivated algae or cyanobacteria. This coupling can either be physical, where the two genes are covalently linked prior to transformation, or by the same physical juxtaposition commonly achieved by co-transformation. Both will heretofore be termed “tandem”, as the result is tightly linked genes. These would render individuals released to natural ecosystems unfit to act as competitors with its own wild type as well as other algae and cyanobacteria species.

In a special case, if the cultivated algae or cyanobacteria are asexual and non-conjugating, it is only necessary to mitigate the effects of self propagation. In that case the genetically engineered, commercially desirable genetic trait can be transformed into a population of the cultivated algae or cyanobacteria that express an irreversible (e.g. deletion) mutation conferring a mitigating trait. Such mutations exist in culture collections or can be obtained by mutagenesis, preferably by ultraviolet or gamma irradiation that causes deletions that cannot be reversed. Chemical mutagenesis, which typically causes point mutations in a single nucleotide can be reversed. Such mutations have been reported, e.g. in chloroplast antenna (Melis et al. 1998, Lee et al., 2002), with reduced RUBISCO (Khrebtukova and Spreitzer. 1996), lacking organs of motility (Okamoto and Ohmori 2002; Tanner et. al 2008). If the genetically engineered, commercially desirable genetic trait(s) is/are transformed into algae or cyanobacteria bearing such a natural or induced mutation that acts as a mitigating genetic trait, then they will be less fit than native algae or cyanobacteria and will not be able to establish themselves in natural ecosystems.

As further detailed and exemplified herein below, genes that decrease RUBISCO or starch, remove cilia or other movement organelles, among others, would all be useful for that purpose, as they would often be benign or advantageous to the cultivated algae or cyanobacteria while detrimental establishment in the wild.

TABLE 2
Examples of commercially desirable traits that can be engineered into algae and
cyanobacteria that would render algae or cyanobacteria unfit and non-competitive
if released into natural ecosystems in addition to a gene suppressing carbonic
anhydrase synthesis or activity.

| Trait | Genetic Element | Source |
|---|---|---|
| Lowered RUBISCO | Antisense or RNAi of large and/or | Native |
| | small RUBISCO subunit | |
| Decreased starch | Sta-1 | RNAi or antisense endogenous gene |
| Increased inulin | 1-SST, 1FFT | RNAi or antisense endogenous gene |
| Modified flagella or cilia | oda1-12, PilT | Chlamydomonas/ |
| | | Synechococcus sp. PCC 7002 |
| Decreased nitrate | Nitrate reductase and/or nitrite | RNAi or antisense endogenous gene |
| reductase | reductase | |
| Decreased cell wall | polysaccharide synthase | RNAi or antisense endogenous gene |
| Reduced CO₂ | Glycolate dehydrogenase | RNAi or antisense endogenous gene |
| concentrating | | |
| mechanism | | |
| Reduced CO₂ | haloacid dehydrogenase | RNAi or antisense endogenous gene |
| concentrating | | |
| mechanism | | |

A transgene encoding reduced activity of the carbon concentrating mechanism allows algae or cyanobacteria growth only at artificially high carbon dioxide concentrations. The transgenes summarized in Table 2 could further augment the reduced carbonic anhydrase activity.

An antisense or RNAi construct targeting the suppression of any of the genes encoding cilia or flagella (or similar motility organ) formation or action prevents the transgenic alga or cyanobacterium to position itself optimally based on environmental stimuli. Such movement is required to compete in natural ecosystems, but is unnecessary and wastes energy in commercial cultivation.

A transgene encoding one or more of the polymers of the cell wall, in the anti-sense or RNAi form causes the alga or cyanobacterium to form a thinner cell wall. This thinner cell wall is of little consequence in commercial production, and the cell walls are the least commercially valued part of the cell, but organisms with thinner cell walls are less competitive in the variable vicissitudes of environmental conditions in natural ecosystems.

A transgene encoding a storage polymer such as inulin, levan, or graminan that is not degradable for use as energy when needed, especially if coupled with RNAi or antisense form of, for example, starch phosphorylase targeting the suppression of the gene prevents energy storage as starch. This is desirable in commercial production when the new polymer has a greater value than starch, but renders an organism that cannot mobilize reserves, less fit in a natural environment where it cannot compete with organisms that can mobilize reserves in times of need.

A transgene in the anti-sense or RNAi form targeting the reduction of the nitrate reductase gene which catalyzes the last three steps in the reduction of nitrate to NH₄⁺ prevents formation of ammonia. Unless ammonia is supplied exogenously this transgenic cell will not be able to establish in a natural environment.

Any other transgene that is neutral or beneficial to the algae or cyanobacteria when cultivated commercially, but renders the algae or cyanobacteria unfit to compete in natural ecosystems, overcoming any benefit that may derive from the transgene tandemly bound to it may be used as well.

The present invention also provides a genetic construct for mitigating the effects of establishment or introgression of a genetically engineered commercially desirable genetic trait of a cultivated alga or cyanobacterium. The genetic construct comprises a first polynucleotide sequence encoding at least one commercially desirable genetic trait and a second polynucleotide sequence encoding at least one mitigating genetic trait. Expression of the commercially desirable and the mitigating genetic trait is genetically linked. The polynucleotide encoding the first, primary genetic trait is preferably flanked on both sides by polynucleotides encoding the second, mitigating genetic trait, to thereby reduce the risk of losing the second, mitigating genetic trait due to mutation, etc.

However, it will be appreciated that in many cases while using conventional transformation techniques genetic traits carried on two different vectors integrate to the same locus.

Thus, according to a further aspect of the present invention there is provided a cultivated algae or cyanobacteria genetically modified to include the above described genetic constructs and to express the traits encoded thereby.

In one embodiment, a second, mitigating genetic trait is selected from the group consisting of reduced RUBISCO, reduced starch content, reduced nitrate reductase, or removal of cilia or other propelling organelles. Numerous specific examples of such genetic traits are listed herein and are further discussed in the Examples section that follows.

One such mitigating trait is reduced RUBISCO content. Genetically reduced RUBISCO would be neutral or advantageous to the algae or cyanobacteria growing in saturating carbon dioxide, but deleterious to the algae or cyanobacteria in the wild, by themselves or in introgressed progeny, where carbon dioxide is limiting and there would not be enough enzyme to fix carbon dioxide.

Another such mitigating trait is decreased starch content. Such algae or cyanobacteria would be desirable as they would funnel more photosynthates to more valuable products, but without starch, such algae and cyanobacteria would not have the desired storage components to compete and exist in natural ecosystems

Still another such mitigating trait is using mutants that are obtained transgenically and are less mobile.

Thus, these anti-establishment in natural ecosystem, introgression-mitigating traits are combined, according to the present invention, with the desirable genetically engineered traits, which genetically engineered traits include, but are not limited to, traits imposing resistance to herbicides, disease, zooplankton pests, and pathogens, resistance to environmental stress such as, but not limited to, heat, salinity, etc., and traits affecting yield, modified product and by-product quality, bioremediation, as well as expression of heterologous products and genetically modified products such as starches and oils, etc. Such traits for which genes have been isolated are well known in the art, for example, genes modifying fatty acid content [delta(12)-fatty acid dehydrogenase (fad2), fatty acid desaturase, and thioesterase (TE)], PAT), herbicide tolerance genes that collaterally control many bacterial and fungal pathogens (5-enolpyruvylshikimate-3-phosphate synthase (EPSPS), acetolactate synthase, glyphosate oxidoreductase, nitrilase, phosphinothricin N-acetyltransferase) as well as genes conferring favorable mutations (phytoene desaturase (pds), acetolactate synthase (ALS), and acetyl-CoA-carboxylase, protoporphyrinogen oxidase (PPO or protox), glutamine synthetase, and other herbicide resistance genes), and numerous viral resistance genes (helicase, replicase and various specific viral coat protein genes). Additional suitable genes are listed in Table 1 and summarized in many recent publications.

Once a gene responsible for a mitigating trait has been selected, it must be engineered for algal or cyanobacterial expression along with the desirable trait that confers an advantage thereto. To introduce such genes into algae or cyanobacteria, a suitable chimeric gene and transformation vector must be constructed. A typical chimeric gene for transformation will include a promoter region, a heterologous structural DNA coding sequences and a 3′ non-translated polyadenylation site for algae. A heterologous structural DNA coding sequence means a structural coding sequence that is not native to the algae or cyanobacteria being transformed. Heterologous with respect to the promoter means that the coding sequence does not exist in nature in the same orientation with the promoter to that it is now attached. Chimeric means a novel non-naturally occurring gene that is comprised of parts of different genes. In preparing the transformation vector, the various DNA fragments may be manipulated as necessary to create the desired vector. This includes using linkers or adaptors as necessary to form suitable restriction sites or to eliminate unwanted restriction sites or other like manipulations that are known to those of ordinary skill in the art.

Promoters that are known or found to cause transcription of a selected gene or genes in plant and bacterial cells can be used to implement the present invention in algae or cyanobacteria, respectively. Such promoters may be obtained from plants, plant pathogenic bacteria, or plant viruses, and include, but are not necessarily limited to, strong constitutive promoter such as a 35S promoter (Odell et al (1985), a 35S'3 promoter (Hull and Howell (1987)) Virology 86, 482-493) and the 19S promoter of cauliflower mosaic virus (CaMV35S and CaMV19S), the full-length transcript promoter from the figwort mosaic virus (FMV35S) and promoters isolated from plant genes such as EPSP synthase, ssRUBISCO genes. Selective expression in green tissue can be achieved by using, for example, the promoter of the gene encoding the small subunit of RUBISCO (European patent application 87400544.0 published Oct. 21, 1987, as EP 0 242 246). All of these promoters have been used to create various types of DNA constructs that have been expressed in plants. See, for example PCT publication WO 84/02913 (Rogers et al., Monsanto). The particular promoter selected should be capable of causing sufficient expression to result in the production of an effective amount of the respective proteins to confer the traits.

A particularly useful promoter for use in some embodiments of the present invention is the full-length transcript promoter from the figwort mosaic virus (FMV35S). The FMV35S promoter is particularly useful because of its ability to cause uniform and high levels of expression in plant tissues. The DNA sequence of a FMV35S promoter is presented in U.S. Pat. No. 5,512,466 and is identified as SEQ ID NO:17 therein. The promoters used for expressing the genes according to the present invention may be further modified if desired to alter their expression characteristics. For example, the CaMV35S promoter may be ligated to the portion of the ssRUBISCO gene which represses the expression of ssRUBISCO in the absence of light, to create a promoter which is active in leaves but not in roots. The resulting chimeric promoter may be used as described herein. As used herein, the phrase “CaMV35S” or “FMV35S” promoter includes variations of these promoters, e.g., promoters derived by means of ligation with operator regions, random or controlled mutagenesis, addition or duplication of enhancer sequences, etc. Other promoters to be used are actin, tubulin ubiquitin, fcpA, fcpB from various organisms including the endogenous algae as well as cyanobacteria promoters

The 3′ non-translated region contains a polyadenylation signal that functions in algae (but not cyanobacteria) to cause the addition of polyadenylated nucleotides to the 3′ end of an RNA sequence. Examples of suitable 3′ regions are the 3′ transcribed, non-translated regions containing the polyadenylation signal of plant genes like the 7s soybean storage protein genes and the pea E9 small subunit of the RuBP carboxylase gene (ssRUBISCO).

The RNAs produced by a DNA construct of the present invention also preferably contains a 5′ non-translated leader sequence. This sequence can be derived from the promoters selected to express the genes, and can be specifically modified so as to increase translation of the mRNAs. The 5′ non-translated regions can also be obtained from viral RNA's, from suitable eukaryotic genes, or from a synthetic gene sequence. The present invention is not limited to constructs wherein the non-translated region is derived from the 5′ non-translated sequence that accompanies the promoter sequence. Rather, the non-translated leader sequences can be part of the 5′ end of the non-translated region of the native coding sequence for the heterologous coding sequence, or part of the promoter sequence, or can be derived from an unrelated promoter or coding sequence as discussed above.

In a preferred embodiment according to the present invention, the vector that is used to introduce the encoded proteins into the host cells of the algae or cyanobacteria will comprise an appropriate selectable marker. In a more preferred embodiment according to the present invention the vector is an expression vector comprising both a selectable marker and an origin of replication. In another most preferred embodiment according to the present invention the vector will be a shuttle vector, which can propagate both in E. coli (wherein the construct comprises an appropriate selectable marker and origin of replication) and be compatible for propagation or integration in the genome of the algae or cyanobacterium of choice. In yet another embodiment, the construct comprising the promoter of choice, and the gene of interest is placed in a viral vector which is used to infect the cells. This virus may be integrated in the genome of the organism of choice or may remain non-integrated.

According to some embodiments of the present invention, secretion of the protein or proteins out of the cell is preferred. In such embodiments the construct will comprise a signal sequence to effect secretion as is known in the art. For some applications, a signal sequence that is recognized in the active growth phase will be most preferred. As will be recognized by the skilled artisan, the appropriate signal sequence should be placed immediately downstream of the translational start site (ATG), and in frame with the coding sequence of the gene to be expressed.

Introduction of the construct into the cells is accomplished by any conventional method for transformation, transfection, infection with Agrobacterium tumefaciens or the like as is known in the art including electroporation, microporation, and/or biolistic transformations. In constructs comprising a selectable marker the cells may be selected for those bearing functional copies of the construct. If the plasmid comprising the gene of interest is episomal, the appropriate selective conditions will be used during growth. Stable transformants and stable cell lines may be derived from the transformed/transfected cells in appropriate cases, in order to conveniently maintain the genotype of interest. Cell growth is accomplished in accordance with the cell type, using any standard growth conditions as may be suitable to support the growth of the specific cell line.

A DNA construct of the present invention can be inserted into the genome of algae or cyanobacteria by any suitable method. Such methods may involve, for example, the use of liposomes, electroporation, microporation, glass beads (Kindle, 1990), chemicals that increase free DNA uptake such as polyethylene glycol (PEG), vacuum filtration, particle gun technology (biolistic bombardment with tungsten or gold particles; see, for example, Sanford et al., U.S. Pat. No. 4,945,050; McCabe et al. (1988) Biotechnology, 6:923-926). Also see, Weissinger et al. (1988) Annual Rev. Genet., 22:421-477; Datta et al. (1990) Biotechnology, 8:736-740; Klein et al. (1988) Proc. Natl. Acad. Sci. USA, 85:4305-4309; Klein et al. (1988) Biotechnology, 6:559-563 (maize); Klein et al. (1988) Plant Physiol., 91:440-444; Fromm et al. (1990) Biotechnology, 8:833-839; and Tomes et al. “Direct DNA transfer into intact plant cells via microprojectile bombardment.” In: Gamborg and Phillips (Eds.) Plant Cell, Tissue and Organ Culture: Fundamental Methods; Springer-Verlag, Berlin (1995); Hooydaas-Van Slogteren & Hooykaas (1984) Nature (London), 311:763-764; Bytebier et al. (1987) Proc. Natl. Acad. Sci. USA, 84:5345-5349;) and other mechanical DNA transfer techniques, and transformation using viruses. Such techniques include, but are not limited to, microprojectile injection methods or to electroporative methods, as described in detail herein below. Application of the electroporative systems to different species often depends upon the ability to regenerate that particular algal or cyanobacterial species from protoplasts.

An additional advantage of using the tandem-system according to this disclosure including a gene that may have an advantage in natural ecosystems genetically linked with a mitigating gene is that the pair can be chosen in such a manner that one of the pair can have traits that will allow it to be used as a selectable marker, obviating the need for a separate selectable marker. Confirmation of the transgenic nature of the algal or cyanobacterial cells may be performed by PCR analysis, antibiotic or herbicide resistance, enzymatic or mRNA analysis, and/or Southern analysis to verify transformation, as well as western blot analysis to verify expression. Progeny of the initial algal or cyanobacterial strains may be obtained by continuous sub-culturing and analyzed to verify whether the transgenes are heritable. Heritability of the transgene is further confirmation of the stable transformation of the transgene in the algae or cyanobacteria. The transgenic algae or cyanobacteria are then grown and harvested using conventional procedures.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

Reference is now made to the following examples, which together with the above descriptions, illustrate the invention in a non-limiting fashion. The concept of using genetic engineering to mitigate any positive effects transgenes may confer when released from controlled culture conditions into the natural environment, preventing the establishment of the transgenic algae or cyanobacteria and the products they may have from introgression to other species, is based on the following premise: If a transgene construct has in totality a small fitness disadvantage, it will remain localized as a very small proportion of the population. Therefore, gene establishment and flow should be mitigated by lowering the fitness of recipients below the fitness of the wild type so that they will not spread. This concept of “transgenic mitigation” (TM) was proposed for higher plants U.S. Pat. No. 7,612,255 and in a subsequent publication (Gressel, J. 1999), in which mitigator genes are added to the desired primary transgene, which would reduce the fitness advantage to hybrids and their rare progeny, and thus considerably reduce risk. It is now extended to transgenic algae and cyanobacteria specifically in regards to activity of the carbon concentrating mechanism as the mitigating trait.

In plants, the Transgenic Mitigation (TM) approach is based on the facts that: 1) tandem constructs act as tightly linked genes, and their segregation from each other is exceedingly rare, far below the natural mutation rate; and 2) The TM traits chosen are selected to be nearly neutral or favorable to the cultivated crops, but deleterious to non-crop progeny (weeds, etc) due to a negative selection pressure; and 3) Individuals bearing even mildly harmful TM traits will be kept at exceedingly low frequencies in weed populations because weeds typically have a very high seed output and strongly compete amongst themselves, eliminating even marginally unfit individuals (Gressel, 1999). That this approach has been effective in higher plants has been illustrated in the following scientific publications: Al-Ahmad, et al., (2004; 2005; 2006, Al-Ahmad and Gressel, 2006). This approach can be used in algae and cyanobacteria, using other TM genes that would be positive or neutral under cultivation, but deleterious in natural ecosystems.

Basically, in the cases of algae and cyanobacteria, these findings can be extended by tandemly combining almost any commercially useful trait that might spread in natural environments (Table 1) with a gene encoding suppressed activity of the carbon concentrating mechanism, in some cases together with another commercially neutral or advantageous trait that would render organisms unfit to compete in natural ecosystems (Table 2) as demonstrated in the following non-exclusive examples.

### Example 1

### Demonstration that Algae with Suppressed Carbonic Anhydrase can Photosynthesize Normally at Artificially Elevated Levels of Carbon Dioxide but not at Ambient Levels In Natural Ecosystems

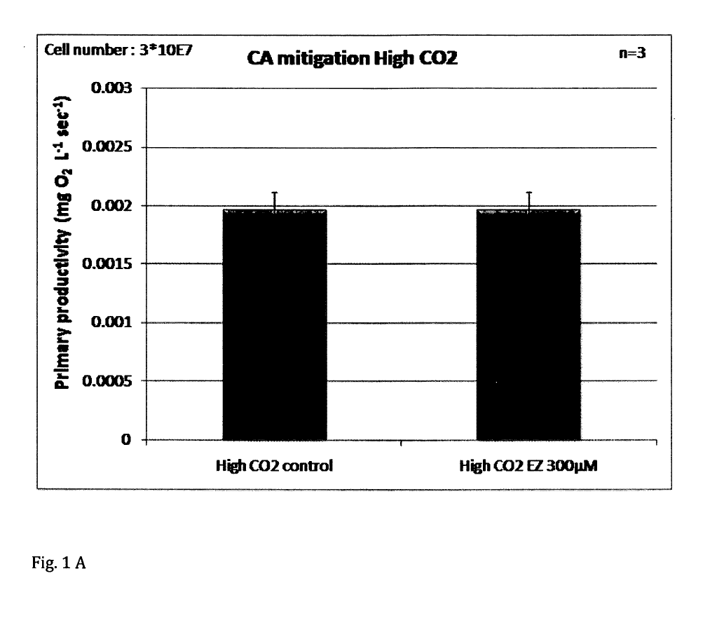

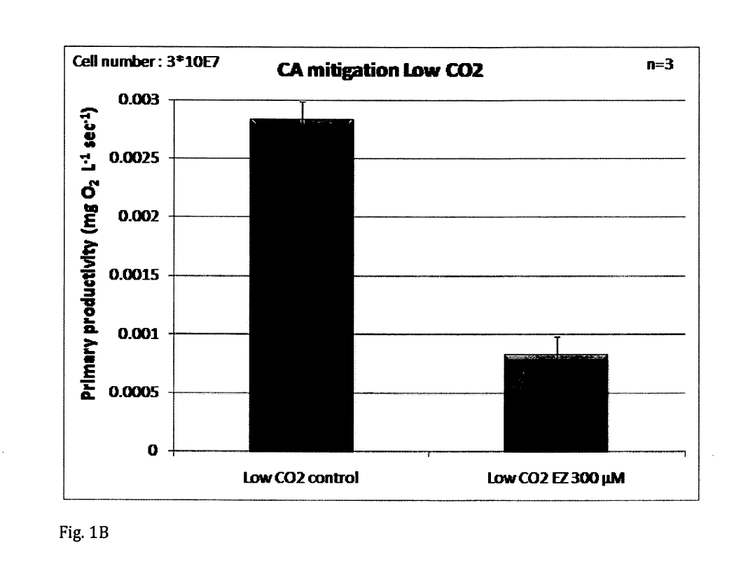

An inhibitor was used to ascertain whether cells inhibited in carbonic anhydrase would be able to photosynthesize normally at artificially high (more than 2%) carbon dioxide levels used in the culture media, and how they would photosynthesize at the low carbon dioxide levels in natural waters. There was no difference in photosynthesis between inhibitor treated and untreated cells when incubated at high carbon dioxide levels (FIG. 1A). The cells were severely deficient in photosynthesis when incubated with the inhibitor at low carbon dioxide levels (FIG. 1B). Thus, transgenic suppression of the carbonic anhydrase enzyme should cut the rate of photosynthesis by a large factor at the ambient CO₂ levels in natural environments. “Escaped” transgenic algae are not able to survive at atmospheric CO₂ levels as they would not be competitive and levels would decline to zero.

### Example 2

### Prevention of Establishment and Introgression of Fluorochloridone Herbicide Resistance by Coupling with Over-Expression of the Porcine Carbonic Anhydrase Protein Inhibitor Conferring Survival on High CO₂ Concentrations

One of the traits suitable for Transgenic Mitigation in constructs with a primary, desirable trait is over-expression of the porcine carbonic anhydrase protein inhibitor (pica), GenBank accession number U36916 (Wuebbens et. al 1997) that inhibits the carbonic anhydrase enzyme. Other similar mammalian genes may also be used. Strains overexpressing carbonic anhydrase protein inhibitor can live only in bioreactors and ponds, where they are exposed to higher CO₂ concentrations, but cannot survive at ambient CO₂ concentrations in natural ecosystems. Rare algae or cyanobacteria introgressing the TM construct could also no longer compete with native organisms in natural ecosystems.

In order to determine whether transformation of a desirable transgene with a mitigator gene would prevent proliferation of transgenic strains having the tandem construct in the case of breach of containment, a tandem construct was made containing an over-expression cassette of the phytoene desaturase gene (SEQ ID NO:1) for fluorochloridone herbicide resistance as the primary desirable gene (GenBank accession # AY639658), and an over-expression cassette of the porcine carbonic anhydrase protein inhibitor (SEQ ID NO: 2) as a mitigator (GenBank accession # U36916), and used to transform Synechococcus PCC7002, Phaeodactylum tricornutum (by particle bombardment), Nannochloropsis oculata CS-179 (by electroporation), Nannochloropsis sp. CS-246 (by electroporation), Nannochloropsis salina (by electroporation or microporation), Isochrysis galbana (by particle bombardment or microporation), Tetraselmis spp (by microporation), Pavlova lutheri CS-182, Nannochloris sp., Synechococcus PCC7942, Synechosystis PCC6803, Chlamydomonas reinhardtii (by glass bead vortexing), Chlorella vulgaris, Chlorella spp as representatives of all algae and cyanobacteria species. The algae come from a large taxonomical cross section of species (Table 3).

TABLE 3
Phylogeny of some of algae used

| Genus | Family | Order | Phylum | Sub-Kingdom |
|---|---|---|---|---|
| Chlamydomonas | Chlamydomonadaceae | Volvocales | Chlorophyta | Viridaeplantae |
| Nannochloris | Coccomyxaceae | Chlorococcales | Chlorophyta | Viridaeplantae |
| Tetraselmis | Chlorodendraceae | Chlorodendrales | Chlorophyta | Viridaeplantae |
| Phaeodactylum | Phaeodactylaceae | Naviculales | Bacillariophyta | Chromobiota |
| Nannochloropsis | Monodopsidaceae | Eustigmatales | Heterokontophyta | Chromobiota |
| Pavlova | Pavlovaceae | Pavlovales | Haptophyta | Chromobiota |
| Isocluysis | Isochrysidaceae | Isochrysidales | Haptophyta | Chromobiota |

Phylogeny according to: http://www.algaebase.org/browse/taxonomy/
Note:
Many genes that in higher plants and Chlorophyta are encoded in the nucleus are encoded on the chloroplast genome (plastome) in the Chromobaiota lower algae (Grzebyk, et al., 2003)

### Assembling the Tandem Construct

Generation of a Chlamydomonas Culture Expressing Phytoene Desaturase (Pds) Together with Over Expression of the Pica Gene Encoding Porcine Carbonic Anhydrase Inhibitor Protein

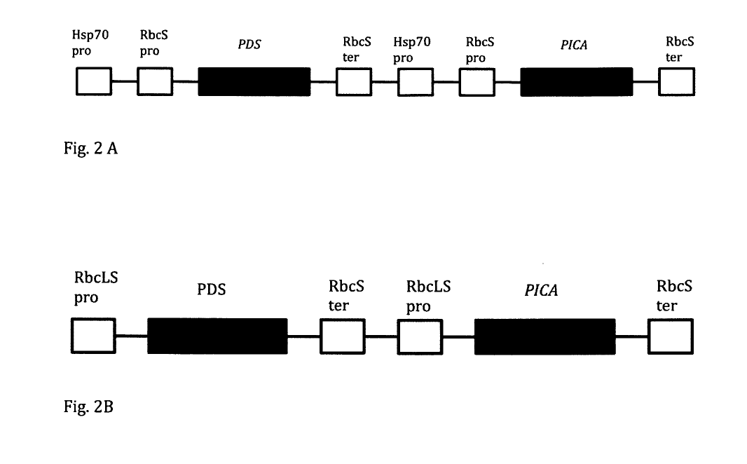

For expression the de novo synthesized phytoene desaturase (pds) gene (SEQ ID NO:1) together with de novo synthesized porcine carbonic anhydrase inhibitor pica (SEQ ID NO:2), which were synthesized according to the codon usage of the desired algae, were cloned under the control of the C. reinhardtii Hsp70-RbcS2 promoter and RrbcS2 terminator and then combined into pSI103 (Sizova, et. al 2001) expression vector (FIG. 2A). In addition, the construct is cloned into various other expression vectors, allowing a range of expression levels driven by different promoters, including constitutive, inducible, and log phase temporal promoters.

### Generation of a Synechococcus PCC7002 Culture Expressing Phytoene Desaturase Together With Over Expression of the Pica Gene

For cyanobacteria, the de novo synthesized pds gene (SEQ ID NO:1) together with de novo synthesized pica (SEQ ID NO:2) are cloned under the control of the constitutive promoter of the rbcLS operon (Deng and Coleman 1999) in the plasmid pCB4 (FIG. 2B), as well as into various other expression vectors, allowing various levels of expression driven by different promoters, including constitutive, inducible, and log phase temporal promoters.

### Transformation of Chlamydomonas

Algae cells in 0.4 ml of growth medium containing 5% PEG6000 were transformed with the plasmid (1±5 mg) by the glass bead vortexing method (Kindle, 1990). The transformation mixture was then transferred to 10 ml of non-selective growth medium for recovery. The cells were kept for at least 18 h at 25° C. in the light. Cells were collected by centrifugation and plated at a density of 10⁸ cells per 80 mm plate. Chlamydomonas transformants were selected on fresh SGII agar plates containing 10⁻⁷M fluorochloridone, for 7-10 days at 25° C.

### Transformation of Marine Algae by Particle Bombardment

Cultures of marine algae are grown in artificial sea water (ASW)+f/2 media until they reach a density of 10⁶ cells/ml. The cells are then centrifuged (2500 g, 10 min, room temp) and washed twice with fresh ASW media. After washing, the cells are re-suspended in an appropriate volume to reach a cell density of 10⁸ cells/ml. 0.5 ml of this cell suspension in then spotted into the center of a 55 mm Petri dishes containing ASW+f/2+15 mM HCO₃⁻ (solidified by 1.5% Bacto-Agar). The Petri dishes are incubated for 24 hrs under standard growth conditions. 0.7 micron tungsten particles (M-10 tungsten powder, Bio-Rad), 0.6 micron gold particles (Bio-Rad) or tungsten powder comprised of particles smaller than 0.6 microns (FWO6, Canada Fujian Jinxin Powder Metallurgy Co., Markham, ON, Canada) are prepared according to the manufacturer's instructions and coated with linear DNA using CaCl₂ and spermidine. Particles are then placed onto macrocarriers and bombarded onto the cells using the Biolistic PDS-1000/He unit (BioRad), 1100 psi rupture discs. This method was adopted, with changes, from Kroth (2007). After bombardment the cells are placed in the growth room for 24 hrs then transferred to a fresh Petri dish containing ASW+f/2+15 mM HCO₃⁻ and a selection agent under standard growth conditions. Colonies of transformed cells appear after 2-3 weeks. Conditions are modified for each organism according to its needs, based on modifications of standard protocols.

### Transformation of Marine Algae by Electroporation

Cultures of Nannochloropsis are grown in ASW+f/2 media for a few days, until they reach a density of 10⁶ cells/ml. To form protoplasts, cells are centrifuged (2500 g, 10 min, room temp) and washed twice with fresh ASW media. After washing, the cells are resuspended in fresh ASW containing 4% hemicellulase (Sigma) and 2% Driselase (Sigma) and incubated in the dark for 4 hrs. Following incubation protoplasts are washed twice (5 min centrifuge, 400 g, room temp) with ASW containing 0.6M sorbitol and 0.6M mannitol (Sigma). Protoplasts are resuspended in an appropriate volume to reach a density of 10⁸ protoplasts/ml. 100 μl of protoplasts are incubated with 10 μg of linear DNA in a 0.1 cm electroporation cuvette (BioRad) on ice for 5 minutes. The protoplasts are then pulsed using the ECM 830 (BTX) electroporator. A series of pulse conditions are applied, ranging between 1000-1400 volts, 6-10 pulses, 10-20 ms each pulse. Samples are then placed immediately on ice for 10 minutes. Protoplasts are transferred to fresh liquid ASW+f/2 media and placed under standard growth conditions for 24 hrs. The treated protoplasts are then transferred to a fresh Petri dish containing ASW+f/2+15 mM HCO₃⁻ and a selection agent and placed under standard growth conditions. Colonies of transformed cells appear after 2-3 weeks.

Conditions are modified for each organism according to its needs, based on modifications of standard protocols.

### Transformation of Marine Algae by Microporation

A fresh algal culture is grown to mid exponential phase in ASW+f/2 media. A 10 ml sample of the culture is harvested, washed twice with Dulbecco's phosphate buffered saline (DPBS, Gibco, Invitrogen, Carslbad, Calif., USA) and resuspended in 250 μl of buffer R (supplied by Digital Bio, NanoEnTek Inc., Seoul, Korea, the producer of the microporation apparatus and kit). After adding 8 μg linear DNA to every 100 μl cells, the cells are pulsed. A variety of pulses is usually needed, depending on the type of cells, ranging from 700 to 1700 volts, 10-40 ms pulse length; each sample is pulsed 1-5 times. Immediately after pulsing the cells are transferred to 200 μl fresh growth media (without selection). After incubating for 24 hours in low light at 25° C., the cells are plated onto selective solid media and incubated under normal growth conditions until single colonies appear.

### Agrobacterium-Mediated Transformation of Marine Algae

Cultures of marine algae are grown in ASW+f/2+HCO₃⁻ media for a few days, until they reach a density of 10⁶ cells/ml. Approximately 10⁶ algae cells are plated on solid ASW+f/2 media in Petri dishes and incubated under normal growth conditions until a lawn of cells is observed. Agrobacterium (A₆₀₀=0.5) bearing the appropriate plasmid (pCAMBIA1301 containing the gene of interest, see Kathiresan et al., 2009) is grown overnight in liquid LB medium then harvested by centrifugation at 3000 g for 10 min. The pellet is resuspended in ¼ASW+f/2 medium. A 200 aliquot of the bacterial culture is then plated on a lawn of marine algae and the plates are incubated under normal growth conditions. After 48 h the cells are harvested and washed with ASW+f/2 containing 200 μg/ml augmentin to kill the Agrobacterium. The algae cells are recovered by centrifugation, washed, and then transferred to a fresh Petri dish containing ASW+f/2+15 mM HCO₃⁻ and a selection agent under standard growth conditions. Colonies of transformed cells appear after 2-3 weeks. Conditions are modified for each organism according to its needs, based on modifications of standard protocols.

### Transformation of Cyanobacteria

For transformation of Synechococcus PCC7002, cells are cultured in 100 ml of BG11+Turk Island Salts liquid medium (http://www.crbip.pasteur.filfiches/fishemedium.jsp?id=648) at 28° C. under white fluorescent light and subcultured at the mid-exponential phase of growth. To 1.0 ml of cell suspension containing 2×10⁸ cells, which are cultured at the mid-exponential phase of growth, 0.5 or 1.0 μg of donor DNA (in 10 mM Tris/1 mM EDTA, pH 8.0) is added, and the mixture is incubated in the dark at 26° C. overnight. After incubation for a further 6 h in the light, the transformants are directly selected on BG11+ Turk Island Salts solid media containing 1.5% agar, 1 mM sodium thiosulfate and a selection agent. The transformation frequency is calculated by counting the number of transformants.

### Gene Integration Analyses of Algal or Cyanobacterial Transformants

Genomic DNA is isolated using either Stratagene's (La Jolla, Calif., USA) DNA purification kit or a combination of QIAGEN's (Valencia, Calif., USA) DNeasy plant mini kit and phenol chloroform extraction (Porebski, et. al 1997). Total RNA is isolated using either QIAGENS's Plant RNeasy Kit or the Trizol Reagent (Invitrogen, Carlsbad, Calif., USA).

The DNA is analyzed by PCR for the presence of intact tandemly linked pds and pica genomic insert. Two different DNA segments within the genomic TM T-DNA insert are amplified with the following primers:

pds forward primer 1 (SEQ ID NO: 3):
ATGACTGTTGCTAGGTCGGT
pds reverse primer 2 (SEQ ID NO: 4):
TCGTCAACGTCTGTGGGCTT
pica forward primer 1 (SEQ ID NO: 5):
TGCGTCTTGCTGTGCGCGGG
pica reverse primer 2 (SEQ ID NO: 6):
TGGAAAGTGCAGGCATCCAG

PCR reactions are carried out in 50 μL aliquots containing about 200 ng genomic DNA, 5 μL, of 10× DyNAzyme™ II buffer (Finnzymes Oy, Espoo, Finland), 1.5 U of DyNAzyme™ II DNA polymerase (Finnzymes Oy, Espoo, Finland), 5 μL of 2.5 mM of each dNTP(s) (Roche Diagnostics, GmbH), and 35 pmol of each primer, in sterile distilled water. The mixture was denatured for 3 min at 94° C. and amplified for 35 cycles (94° C. for 30 s, 50° C. for 30 s, 72° C. for 2 min) with a final cycle of 7 min at 72° C. The PCR products (15 μL) are loaded directly onto 1% (w/v) agarose gels to verify single bands. The remaining PCR products are purified using the QIAquick PCR Purification Kite (Qiagen, Hilden, Germany) according to the manufacturer's instructions, and sequenced to confirm the integration of the TM T-DNA.

In vivo pds assay. Putative transformed algal or cyanobacterial cells were cultured in a solution of 0.1 μM fluorochloridone in standard algae or cyanobacteria culture media. At this concentration, all non-transgenic cells are killed.

In vivo pica assay Picked fluorochloridone resistant algae or cyanobacteria cells are screened for inhibition of carbonic anhydrase activity. Inhibition of carbonic anhydrase activity is measured by preincubating the inhibitor sample and carbonic anhydrase in the colorimetric buffer at 25° C. for at least 1 min, diluting 2.5-fold into CO₂-saturated water, and then assaying carbonic anhydrase activity (Roush & Fierke, 1992). The best inhibition activity possessing colonies are chosen.

Competition of TM transgenics with the wild type algae and cyanobacteria The transgenic TM algae or cyanobacteria are used to compete with natural species in simulated conditions. 1000 transgenic cells per ml were pipetted into unfiltered sea water in aquaria. Aliquots are removed initially at daily, and later at weekly intervals and were plated on 0.1 μM fluorochloridone supplemented with 1.5% CO₂. Within a few weeks, no fluorochloridone resistant and CA inhibitory colonies are found in the aquaria.

### Example 3

### Isolation of Algal Carbonic Anhydrase by Reverse Genetics

Algal cultures are grown under high (at least 1% in air) and ambient (0.039%) CO₂ conditions for 24 hours. Proteins from these cultures are isolated utilizing a buffer containing 750 mM, Tris-HCl pH 8.0, 15% sucrose (wt/vol), 100 mM β-mercaptoethanol and 1 mM phenylmethylsulfonylfluoride (PMSF). Samples are then centrifuged for 20 min at 13,000 g at 4° C., with the resulting proteins separated by 2 dimensional gel electrophoresis (Görg et. al 2000). The first dimension separates proteins by isoelectric focusing according to their pI value (using a pH range of 3-10), and the second dimension separates proteins according to their size (12% PAGE). Protein spots that appear to be induced under ambient, but not high CO₂ conditions are excised, digested by trypsin, analyzed by LC-MS/MS on DECA/LCQ and identified by Pep-Miner and Sequest software against all non redundant databases. The sequencing stages are carried out at The Smoler Proteomic Research Center, Technion—Israel Institute of Technology. All protein sequences are identified according to their homology to known protein sequences in the database. As carbonic anhydrases are known to be induced under ambient CO₂ conditions, some of the sequenced proteins will have a degree of homology to known carbonic anhydrases. The DNA coding sequence is then deduced from the protein sequence, and PCR primers are designed accordingly. Using genomic DNA from the algal wild type cultures as a template, the primers are used to obtain the gene encoding for the desired carbonic anhydrase. Specific RNAi is designed to down-regulate the carbonic anhydrase (see example 4), and using known transformation techniques transformants defective in carbonic anhydrase activity are selected (see example 2).

### Example 4

### Generation of Transgenic Isochrysis Galbana Expressing RNAi Cassette for Carbonic Anhydrase (GenBank ACCESSION: AY826841)

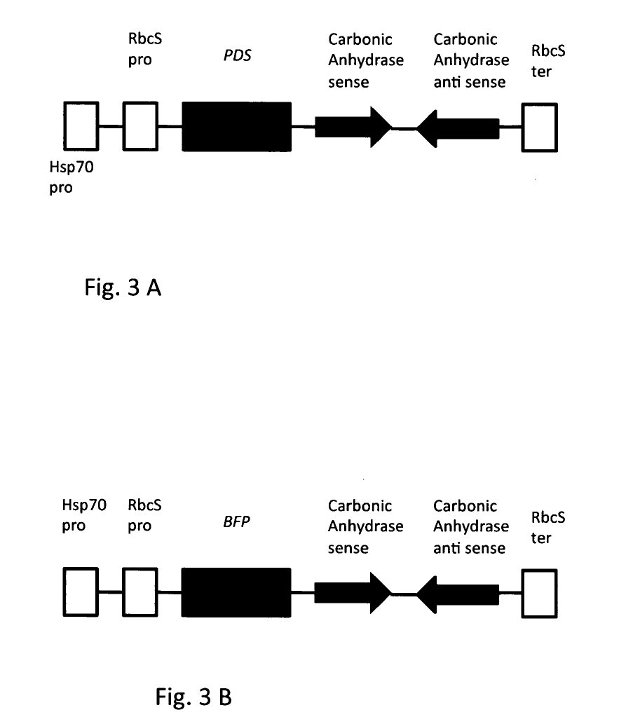

For generation of RNAi of Isochrysis galbana carbonic anhydrase gene (GenBankACCESSION: AY826841) (SEQ ID NO: 7), a 240 bp fragment corresponding to the coding sequence of carbonic anhydrase (nucleotides 1 to 240) is chemically synthesized in both orientations. The two complementary fragments are separated by an intron from the Chlamydomonas rbcS gene. The construct is designed to produce an RNA containing double-stranded stem and loop The RNAi fragment is cloned downstream to the pds (FIG. 3A) gene which confers resistance to fluorochloridone (See U.S. 61/191,167 incorporated herein by reference) or the blue fluorescence protein (BFP) reporter gene (See U.S. 61/192,447 incorporated herein by reference) (FIG. 3B). The transgene is under the control of the Chlamydomonas Hsp70-RbcS2 promoter and RbcS2 terminator in the plasmid pSI103 (Sizova et al., 2001)

Transformants' resistant to fluorochloridone are tested for CO₂ requirements. Transformants and wild-type cells are grown under ambient (0.03%) and high (4%) CO₂ concentrations. Transformants that grow only on high CO₂ levels are selected for further analysis as described in Examples 8 and 9.

### Example 5

### Isolation of Nannochloris and Nannochloropsis Carbonic Anhydrase Partial Gene Sequences for the Production of a RNAi Cassette

Multiple protein alignments of carbonic anhydrase sequences were used to design degenerate primers towards conserved regions of carbonic anhydrase genes. Two degenerate primers were designed according to this alignment as follows:

| | | |
|---|---|---|
| For | TACYTSTACATCGGBTGCGTBGA | (SEQ ID NO: 8) |
| and | | |
| Rev | GTGGARGCKRTAGACRTCNC | (SEQ ID NO: 9) |

based on the regions:

| | | |
|---|---|---|
| For | YLYIGCVD | (SEQ ID No: 10) |
| and | | |
| Rev | RDVYRLH. | (SEQ ID NO: 11) |

These primers are used to amplify a carbonic anhydrase gene fragment from Nannochloris and Nannochloropsis cDNA. The isolated sequences are designed in RNAi cassette, coupled to phytoene desaturase herbicide resistant gene, as described in Example 4. Transformants resistant to fluorochloridone are tested for CO₂ requirements. Transformants and wild-type cells are grown under low (ambient) and high (4%) CO₂ concentrations. Transformants that grow only on high CO₂ levels are selected for further analysis as described in Examples 8 and 9.

### Example 6

### Overexpression of Arabidopsis Carbonic Anhydrase Protein in Algae Chloroplast

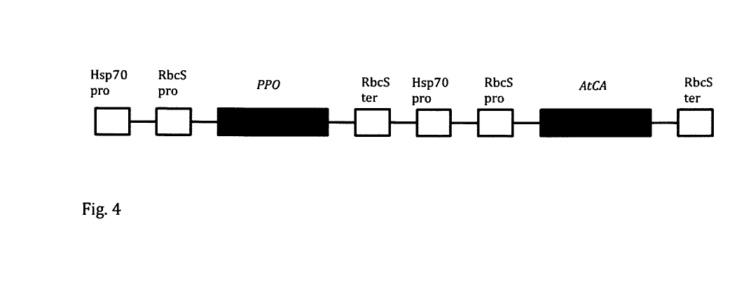

In order to determine whether transformation of a desirable transgene with a mitigator gene would prevent proliferation of transgenic strains having the tandem construct in the case of breach of containment, a tandem construct was made containing an over-expression cassette of the protoporphyrinogen oxidase (ppo) gene (SEQ ID NO:14) for butafenacil herbicide resistance as the primary desirable gene (GenBank ACCESSION NO: ABD52328), tandem with an over-expression of the Arabidopsis βCAII gene (GenBank Accession No. NP 568303). The Arabidopsis chloroplast carbonic anhydrase is chemically synthesized according to Chlamydomonas codon usage (SEQ ID NO: 13) and 3×HA tag is fused to its C′ terminal to enable detection of the transgene. The βCAII gene is cloned under the Hsp70-RbcS2 promoter (Sizova et al., 2001) in tandem with the ppo gene that confers resistance to butafenacil (see U.S. 61/191,167) and is cloned under the same promoters (FIG. 4). Transgenic algae are selected on solid media containing 1 μM butafenacil and colonies with high expression levels of the carbonic anhydrase protein are chosen using western analysis with anti-HA tag antibodies. Transformants resistant to butafenacil that exhibit the best carbonic anhydrase protein expression are tested for CO₂ requirements. Transformants and wild-type cells are grown under ambient (0.03%) and high (4%) CO₂ concentrations. Transformants that grow only on high CO₂ levels are selected for further analysis as described in Examples 8 and 9.

### Example 7

### Overexpression of Synechococcus PCC 7942 Carbonic Anhydrase Protein in Algae Cytosol

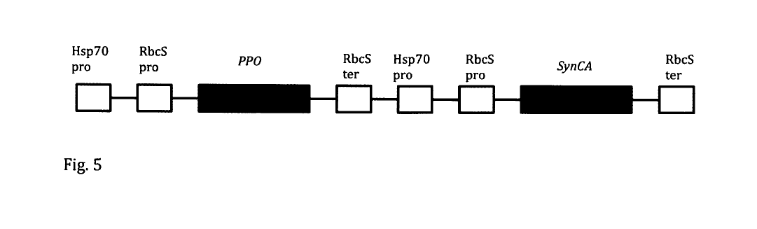

The Synechococcus PCC7942 carbonic anhydrase gene (Accession number: M77095) is chemically synthesized according to Chlamydomonas codon usage (SEQ ID NO: 12) and 3×HA tag is fused to its C′ terminal end to enable detection of the transgene. The cytoplasmatic cyanobacteria carbonic anhydrase gene is cloned under the Hsp70-RbcS2 promoter (Sizova et al., 2001) in tandem with the protoporphyrinogen oxidase (ppo) gene that confers resistance to butafenacil (U.S. 61/191,167) and is cloned under the same promoters (FIG. 5). Transgenic algae are selected on solid media containing 1 μM butafenacil and colonies with high expression levels of the carbonic anhydrase protein are chosen using western analysis with anti-HA tag antibodies. Transformants resistant to butafenacil and exhibit the best carbonic anhydrase protein expression are tested for CO₂ requirements. Transformants and wild-type cells are grown under low (ambient) and high (4%) CO₂ concentrations. Transformants that grow only on high CO₂ levels are selected for further analysis as described in Examples 8 and 9.

### Example 8

### Demonstration that Transformed Algae Exhibit Reduced Photosynthetic Activity at Ambient CO₂ Levels, while Unaffected at Elevated CO₂ Levels

Cultures of down-regulation or over-expression of carbonic anhydrase transformants of algae are compared to wild-type cells. They are cultured initially under high (4%) carbon dioxide and then transferred to ambient CO₂ concentrations and reduced photosynthetic rates are seen as the carbon dioxide is depleted, compared to with wild type cells, which continue to evolve oxygen.

### Example 9

### Demonstration that Transformed Algal Strains Cannot Compete with Wild Type Strains at Ambient CO₂ Concentrations

The transformations described above all enable the algal transformants to function best under bioreactor/pond conditions, namely high CO₂ concentrations. An additional benefit arising from this condition-related culturing is that these strains cannot cope with naturally occurring conditions such as ambient CO₂ concentration. Being currently at 0.03% in the atmosphere, CO₂ becomes a major limiting factor for the transformed strains that are subjected to CO₂ leakage due to over expression of carbonic anhydrase. In order to demonstrate such growth limitation, the carbonic anhydrase transformants are co-cultured with wild-type cells at ambient CO₂ concentrations. A time-sequence sampling protocol is followed with collected cells from the growth vessel. Cells are then transferred to plates for colony isolation (single cell plating and replica plating on dishes) and at the right dilution plates are duplicated. One plate contains normal growth media while its duplicate contains a selection factor (e.g. herbicide fluorochloridone). This enables differentiation between wild-type cells and transformants. The wild-type cells outcompete carbonic anhydrase transformants in a few generations.

### The Methodologies Used in the Various Steps of Enabling the Invention:

RNA Extraction, cDNA Synthesis and Quantitative RT-PCR Analysis

Total RNA is isolated using either QIAGENS's Plant RNeasy Kit (QIAGEN, Hilden, Germany) or the Trizol Reagent (Invitrogen, Carlsbad, Calif., USA). cDNA is synthesized using 3 μg total RNA as a template with oligo-dT primer and AMV reverse transcriptase (CHIMERx Milwaukee, Wis., USA) according to the manufacturer's instructions. This is used to test isolated carbonic anhydrase sequences for inducibility at low carbon dioxide concentrations. Real-time quantitative PCR reactions are performed in an optical 96-well plate using the ABI PRISM 7300 Sequence Detection System (Applied Biosystems, Scoresby, Victoria, Australia) and SYBR Green I for monitoring dsDNA synthesis. For all PCR reactions the following standard thermal profile was used: 50° C. for 2 min; 95° C. for 15 min; 40 cycles of 95° C. for 15 sec and 60° C. for 1 min. In order to compare data from different cDNA samples, C_{T} (threshold cycle) values for all genes are normalized to the C_{T} values of ubiquitin, or 16S rDNA which are used as internal references in all algal and cyanobacterial experiments respectively. All primers are designed using the Primer Express 2.0 software (Applied Biosystems). The real-time PCR data are analyzed using the comparative CT-method with appropriate validation experiments performed in advance (Applied Biosystems, User Bulletin #2, http://home.appliedbiosystems.com/). All experiments are repeated at least three times with cDNA templates prepared from three independent colonies of algae or cyanobacteria and every reaction was set up in duplicates. The algae were transformed as described in Example 2.

### Physiological Assessment

To assess physiological properties of genetically modified algae compared with their relevant wild type strains we performed a set of procedures that enabled us to evaluate each strain. Initially, each genetically modified strain is checked for the trait modified, as explained above. Next, the fastest growing colonies are selected and transferred to liquid medium for further physiological evaluation. This includes measurement of: growth rate, photosynthetic activity, respiration activity, tolerance to abiotic parameters, lipid content and protein content.

### Growth Rate

Growth rates are measured using one or more of the following techniques:
Direct cell count
Optical density at a relevant wavelength (e.g. 750 nm)
Pigment/chlorophyll concentration (where this method is applicable)
Dry weight

### Photosynthetic Activity

One of the important parameters indicating the welfare of a photoautotrophic culture is its photosynthetic capability. Photosynthetic activity is monitored by measuring oxygen evolution and/or by variable fluorescence measurements:

We also evaluate oxygen consumption in the dark in order to estimate net photosynthetic potential of the algal culture. As part of the photosynthetic evaluation we follow several abiotic parameters that potentially influence the physiological state of a culture.
Light intensity tolerance (at a given cell density) is evaluated. P/I (photosynthesis vs. irradiance) curves are used to determine optimal light intensity per cell.
Performance at different CO₂ levels (e.g. ambient; 1%; 5%). This is coupled with pH tolerance.
Temperature tolerance. Each culture is tested at its optimal temperature for growth. In addition, temperatures are raised gradually and culture activities (as described above) are measured.

### Growth Conditions

Cells of eukaryotic marine cultures (e.g. Chlorella vulgaris, Phaeodactylum tricornutum, Isochrysis sp., Nannochloris spp. and Nannochloropsis sp.) and transformants thereof are grown on artificial seawater medium (Goyet and Poisson, 1989) supplemented with f/2 (Guillard and Ryther, 1962). Marine cultures are grown at 18-22° C. with a 16/8 h light/dark period. Fresh water cultures (e.g. Chlamydomonas reinhardtii) and mutants thereof are grown photoautotrophically on liquid medium, using mineral medium as described in (Harris, 1989), with the addition of 5 mM NaHCO₃⁻, with continuous shaking and illumination at 22° C.

### Growth Rate Estimation

Cells are harvested in the logarithmic growth phase and resuspended in fresh growth media. Cultures are brought to a cell density corresponding to ˜3 μg/ml chlorophyll a. Light intensity is optimized for each culture and temperature is maintained at growth temperature ±1° C. Where required, cells are concentrated by centrifugation (3000 g, 5 min) and resuspended in a fresh media. A time-series sampling procedure is followed where a subsample of each culture is collected and the number of cells per ml measured. Direct counting, optical density at different wavelengths, packed volume at stacked assay and chlorophyll concentrations are also measured.

### Oxygen Evolution

Measurements of O₂ concentrations are performed using a Clark type O₂ electrode (Pasco Scientific, Roseville, Calif., USA). 20 ml of cell suspension corresponding to 15 μg chlorophyll/ml are placed in the O₂ electrode chamber, at relevant temperature. Cells are exposed to various light intensities and net O₂ production is measured. Dark incubations are performed in air-tight vessels to follow light-independent O₂ consumption.

### Fluorescence Measurements

Electron transfer activity of photosystem II is measured by pulse modulated fluorescence (PAM) kinetics using PAM-101 (Walz, Effertlich, Germany). Light intensity (measured at the surface of the chamber) of the modulated measuring beam (at 1.6 kHz frequency) is 0.1 μmol photons m⁻²s⁻¹. White actinic light is delivered at 50-1500 μmol photons m⁻² s⁻¹ as required in different experiments and is used to assess steady state fluorescence (Fₛ). Maximum fluorescence (Fₘ) is measured with saturating white light pulses of 4000 μmol photons m⁻² s⁻¹ for 1 s.

### Additional Experiments

Light intensity tolerance (at a given cell density) is evaluated. P/I (photosynthesis vs. irradiance) curves are used to determine optimal light intensity per cell. 20 ml of cell suspension corresponding to 15 μg chlorophyll/ml are placed in the O₂ electrode chamber, at relevant temperature and various light intensities. Oxygen evolution rates are measured at different light intensities.
Performance at different CO₂ levels (e.g. ambient; 1%; 5%). Growth rate estimations and photosynthetic activity (methodology described above) are evaluated when cultures are maintained at different CO₂ levels.
Temperature tolerance. Each culture is tested at its optimal temperature. In addition, we attempt to raise temperatures to the highest point possible without inhibiting growth.

### REFERENCES

Al-Ahmad H., and Gressel J. (2006) Mitigation using a tandem construct containing a selectively unfit gene precludes establishment of Brassica napus transgenes in hybrids and backcrosses with weedy Brassica rapa. Plant Biotechnology Journal 4:23-33.
Al-Ahmad, H. I., S. Galili and J. Gressel (2004) Tandem constructs to mitigate transgene persistence: tobacco as a model. Molecular Ecology 13:697-710
Al-Ahmad, H., S. Galili, and J. Gressel (2005) Poor competitive fitness of transgenically mitigated tobacco in competition with the wild type in a replacement series. Planta 222:372-385
Al-Ahmad H., Dwyer J., Moloney M., and Gressel J. (2006) Mitigation of establishment of Brassica napus transgenes in volunteers using a tandem construct containing a selectively unfit gene. Plant Biotechnology Journal 4:7-21
Deng M D, Coleman J R (1999) Ethanol synthesis by genetic engineering in cyanobacteria. Appl Environ Microbiol 65: 523-528
Görg A, Obermaier C, Boguth G, Harder A, Scheibe B, Wildgruber R and Weiss W (2000) The current state of two-dimensional electrophoresis with immobilized pH gradients, Electrophoresis 21,1037-1053
Goyet, C and A Poisson, 1989, New determination of carbonic acid dissociation constants in seawater as a function of temperature and salinity, Deep-Sea Res. 36: 1635-1654.
Gressel, J. 1999: Tandem constructs: preventing the rise of superweeds. Trends Biotech. 17: 361-366.
Gressel, J., 2008a Genetic Glass Ceilings, Transgenics for Crop Biodiversity, Johns Hopkins University Press, Baltimore.
Gressel, J. (2008b) Transgenics are imperative for biofuel crops. Plant Science 174: 246-263.
Grzebyk, D., O, Schofield, P. Falkowski, and J. Bernhard (2003) The Mesozoic radiation of eukaryotic algae: the portable plastid hypothesis. J. Phycol. 39:259-267)
Guillard, R. R. and Ryther, J. H. 1962. Studies on marine planktonic diatoms. I. Cyclotella nana Hustedt and Detonula confervacaea (Cleve) Gran. Canadian Journal of Microbiology 8: 229-239
Harris, E. H., 1989. The Chlamydomonas Sourcebook: a Comprehensive Guide to Biology and Laboratory Use, Academic Press, San Diego, Calif.
Kathiresan, S., Chandrashekar, A., Ravishankar G. A. and R. Sarada (2009) Agrobacterium-mediated transformation in the green alga Haematococcus pluvialis (Chlorophyceae, Volvocales). J. Phycol. 45:642-649
Khrebtukova, I. and R. J. Spreitzer, 1996. Elimination of the Chlamydomonas gene family that encodes the small subunit of ribulose-1,5-bisphosphate carboxylaseoxygenase. Proc. Natl. Acad. Sci. USA 93: 13689-13693
Kindle K L (1990) High-frequency nuclear transformation of Chlamydomonas reinhardtii. Proceedings of the National Academy of Sciences of the United States of America 87: 1228
Kroth (2007) Genetic transformation: a tool to study protein targeting in diatoms. Methods Mol. Biol. 390:257-67).
Lee, J. W. L. Mets and E. Greenbaum, 2002. Improvement of photosynthetic CO₂ fixation at high light intensity through reduction of chlorophyll antenna size. Applied Biochemistry and Biotechnology 98-100: 37-48.
Melis, A., J. Neidhardt, J. R. Benemann, 1998. Dunaliella salina (Chlorophyta) with small chlorophyll antenna sizes exhibit higher photosynthetic productivities and photon use efficiencies than normally pigmented cells, J. Appl. Phycol. 10: 515-525.
Okamoto S, Ohmori M (2002) The Cyanobacterial PilT Protein Responsible for Cell Motility and Transformation Hydrolyzes ATP. Plant and Cell Physiology 43: 1127-1136
Porebski, S., L. G. Bailey, and B. R. Baum 1997 Modification of a CTAB DNA extraction protocol for plants containing high polysaccharide and polyphenol components. Plant Molecular Biology Reporter 15: 8-15
Roush, E. D. and C. A. Fierke 1992 Purification and characterization of a carbonic anhydrase II inhibitor from porcine plasma Biochemistry 31: 12536-12542
Sheehan, J., et al. (2004). A Look Back at the US Department of Energy's Aquatic Species Program: Biodiesel from Algae; Close-Out Report, Island Press.
Sivan, A. and S. Arad 1998 Intraspecific transfer of herbicide resistance in the red microalga Porphyridium sp. (Rhodophyceae) via protoplast fusion. J. Phycol. 34, 706-711
Sizova, I. M. Fuhrmann and P. Hegemann 2001 A Streptomyces rimosus aphVIII gene coding for a new type phosphotransferase provides stable antibiotic resistance to Chlamydomonas reinhardtii Gene 277: 221-229
Tanner C A, Rompolas P, Patel-King R S, Gorbatyuk O, Wakabayashi K I, Pazour G J, King S M (2008) Three members of the LC8/DYNLL family are required for outer arm dynein motor function. Mol Biol Cell 19:3724-3734.
Wuebbens, M. W. E. D. Roush, C. M. Decastro, and C. A. Fierke 1997 Cloning, sequencing, and recombinant expression of the porcine inhibitor of carbonic anhydrase: a novel member of the transferrin family. Biochemistry 36: 4327-4336

What is claimed is:

1. A method to mitigate the effects of introgression of a genetically engineered advantageous genetic trait of cultivated algae or cyanobacteria to its wild type or to an interbreeding related species such that a mitigated algae or cyanobacteria cannot establish populations outside of cultivation, said method comprising the steps of:
a) introducing into the algae or cyanobacteria genome at least one gene encoding the advantageous trait in tandem with at least one gene encoding a mitigating trait;
said at least one mitigating trait comprising suppressed activity of carbon concentrating mechanism; and
b) cultivating the algae or cyanobacteria under above-ambient CO₂ concentrations whereby the suppressed activity of carbon concentrating mechanism does not affect photosynthesis, and the algae or cyanobacteria carrying the low carbon concentrating mechanism activity die outside of cultivation as a result of insufficient CO₂ concentrating capacity.

2. The method of claim 1, wherein the suppressed activity of carbon concentrating mechanism is achieved by low carbonic anhydrase activity or production in pyrenoids or carboxysomes, or by over-expression of carbonic anhydrase in cytoplasm or in chloroplasts.

3. The method of claim 2, wherein the suppressed activity of carbon concentrating mechanism is achieved by over-expression of cytosolic carbonic anhydrase in cytosol.

4. The method of claim 3, wherein the cytosolic carbonic anhydrase is from Synecococcus PCC 7942.

5. The method of claim 2, wherein the suppressed activity of carbon concentrating mechanism is achieved by over-expression of chloroplast carbonic anhydrase in chloroplasts.

6. The method of claim 5, wherein the chloroplast carbonic anhydrase is from Arabidobis thaliana targeted with its endogenous chloroplastic signal peptide and with exogenous signal peptides.

7. The method of claim 6, wherein the signal peptide is selected from the group consisting of rubisco and phytoene desaturase chloroplastic signal peptides

8. The method of claim 1, wherein the gene encoding a mitigating trait is a gene encoding a carbonic anhydrase protein inhibitor.

9. The method of claim 8, wherein the gene encoding a mitigating trait is gene coding for a porcine carbonic anhydrase protein inhibitor.

10. The method of claim 8, wherein the gene encoding the advantageous trait and the gene encoding a carbonic anhydrase protein inhibitor are expressed under a strong constitutive promoter.

11. The method of claim 10, wherein the promoter is selected from the group consisting of CaMV35S promoter, CaMV19S promoter, FMV35S promoter, Hsp70 promoter ssRubisco promoter, Hsp-Rbcs, and algae endogenous promoters.

12. The method of claim 2, wherein the low carbonic anhydrase activity or production is conferred by antisense or RNAi constructs of the carbonic anhydrase gene.

13. The method of claim 1, wherein the cultivated algae or cyanobacteria can propagate only asexually and the genes of step a) are transformed separately.

14. The method according to claim 1, wherein the advantageous trait is selected from the group consisting of improved fatty acid composition, enhanced photosynthesis, increased methionine content, increased lysine content, anti-microbial resistance, secondary metabolite production, biotransformation of exogenous substrates, herbicide resistance, mercury volatilization and virus- and phage resistance.

15. The method according to claim 1, wherein a second gene encoding for a mitigating trait is included.

16. The method according to claim 15, wherein the second gene encoding for a mitigating trait is selected from the group consisting of genes encoding lowered RUBISCO activity, reduced nitrate reductase, decreased starch accumulation, increased inulin accumulation, modified cilial or flagellar movement and modified cell wall polysaccharide synthesis, along with the, reduced carbon concentrating mechanism related gene.

17. The method according to claim 1, wherein the alga is selected from the group of algal strains consisting of:
Nannochloropsis sp. CS 246, Nannochloropsis oculata, Phaeodactylum tricornutum, Nannochloropsis salina, Pavlova lutheri CS182, Chlamydomonas reinhardtii, Isochrysis spp. Tetraselmis spp., Nannochloris spp., and Chlorella spp.

18. The method according to claim 1, wherein the cyanobacterium is selected from the group of cyanobacterial strains consisting of: Synechococcus PCC 7942, Synechococcus PCC7002, and Synechocystis PCC6803.


SEQUENCE LISTING (14 sequences)

SEQ ID NO: 1 - DNA, 1740 bp, Hydrilla sp
  misc_feature (1)..(1740)
atgactgttgctaggtcggtcgttgcagtcaatctaagtggttcccttcaaaacagatacccagccagttcatcagtcagctgcttccttggcaaagagtacagatgcaacagtatgttaggattctgcggtagtggaaaattggcttttggcgcaaatgcaccctattctaagattgcagctaccaaaccaaagcccaaacttcgccctttgaaggtcaactgcatggatttcccaagacctgatatagataacactgctaatttcttggaagctgctgctctttcttcctcttttcgcaattcagcaagaccaagtaaacctcttcaagttgtaattgctggtgcaggtttggctggtctttcaacagcaaagtatctcgcagatgcagggcacatacccatactactggaggctagagatgtattgggtggcaaggtggcagcgtggaaagatgatgatggagactggtatgagacaggcctgcatatattttttggtgcatatcccaatgtgcagaatttatttggtgaacttggcataaatgatcgtctacaatggaaagagcattcaatgatttttgcgatgccaaacaagccaggggaatttagtcgctttgattttccagaagtacttcctgctccactaaatggaatatgggcaatccttaaaaacaatgaaatgctcacttggccagagaaagtgcaatttgctattggactactacctgcaatgattggggggcagccatatgttgaagctcaggatggcttaacagttcaagagtggatgagaaaacagggtgtgccggatcgagtcaatgacgaggttttcattgcaatgtcaaaggctcttaacttcataaaccctgatgaactttccatgcaatgcatcctgattgccttaaaccatttccttcaggaaaagcatgggtcgaagatggcctttttagatggtaatccacctgaaagattatgtaagccaattgctgatcacatcgagtcattgggtggccaagtcatccttaattcccgaatacagaagattgagctgaatgcagacaaatccgtcaagcattttgtgctcaccaatggaaatataataacaggagatgcatatgtatttgcaacacctgttgatatcttgaagcttctgttacctgaagattggaaggagatttcatatttcaaaaaattggacaagttggttggcgtacctgtgataaatgtacacatatggtttgataggaagttgaagaacacatacgatcatcttcttttcagcaggagtccactgttgagcgtttatgcagacatgtctgttacatgcaaggaatactacaatccaaatcaatccatgcttgagctagtatttgcaccagcagagaaatggatttcatgcagtgacagtgaaatcattaacgcgactatgcaagagcttgctaaactctttccagatgagatttctgctgatcaaagcaaggccaaaattttgaaatatcatgttgtaaagaccccgaggtcagtttacaagacggtccctgattgtgaaccatgccggcctttgcaaagatctccaattgaagggttctacttggctggtgactacacaaagcagaagtatttggcctcaatggaaggtgccgtgttatctgggaagctatgtgctcaggcaattgtgcaggactgcagcttgttggcttctagggtacagaaaagcccacagacgttgacgattgcc

SEQ ID NO: 2 - DNA, 2115 bp, Sus scrofa
  misc_feature (1)..(2115): Porcine inhibitor of carbonic anhydrase encoding nucleic acid
atgaggctcgctttctgcgtcttgctgtgcgcggggtccctggggctgtgtctggcttttcctaaggaaactgtgagatggtgcactgtctcaagtcaagaggccagtaagtgctccagttttcgtcacaatatgaaaaaaatccttccagtggaaggtcctcatgtcagctgtgtgaagagaacctcttacctcgagtgcatcagggccatcttggccaatgaagcagatgctgtgaccatcgatggaggtttggtgtttgaggcaggcctggccccctacaacctgaagcctgtcgtggcagaattctatgggtcaaaagatgatccacaaacccactattacgcggtggccgtggtgaagaagggcagtgacttccagctgagccagctccgaggcaagaagtcctgtcacacaggccttggctggtctgctgggtggaacatccccatggggatacttcttcctcctgactcaggggaagaagcagcagccaagttcttctctagcagctgcgtcccctgtgcggaccggatggccttccccaaaatgtgccaactgtgtgcggggaaaggggtggaaaagtgtgcctgctccaaccatgaacggtacttcggctactcgggcgccttcaagtgtctgcaggaagatgttggggacgtggccttcgtgaggcatgtgactgtgttcgaaaacctgcctgacaaggctgacagggaccagtacgagctgctctgcaaggacaacaccaggaggcctgtggatgactacgagaactgctacctggcacaggtcccttcccacgccgttgtggcccggagtgtggatggcaaggaggacttgatctgggagcttctcaaccaggctcaggaaaattttggaaaagacaagtcggcagagttccagctcttcagctcttctcatgggaaggatctcctgtttacagacgcctgccttgggtttttaagagtcccaccgaaaatggacgcgaagctctatctgggatatgagtattttgctgccattcagcatctaaggagagtccaagggacagaagaaccccagagggtgatgtggtgtgcagtaggccagcacgagaggaccaagtgtgacagctggagtgtcctgagcggcggcatcttgaattgtaactcggaggacaccatggaggactgcatcgctgccatcgcgaaaggagaggctgatgctatgagcttggatggaggcttcctctacacagcgggcaagtgtggtctggtgcctgtcctggcagagaactacttgtctcaagatggcaaagagcggtttgggtctaagtgtgtgaatacacctgtggaaggttattatgtcgtggccgtggtcaagaagtcagatgctgacctcacctggaactccctgagaggcaagaagtcctgccacatagctgttggcacttccgcaggctggatcatccccatgggtttcatatacaaccaaaccgggtcctgtaaacttgatgagttcttcagtcaaagctgtgcccctgggtctgatccagagtcccgtctctgtgctctgtgcagtggcagcatcagtgggcagccagcccacacctgtgcccccaacagccacgaggggtaccacggcttcagtggggccctccggtgcctggttgagaagggagacgtggcctttgtgaagcaccccacagtcctgcagaacaccgacggaaggaaccccgaggcctgggcaaaggatctgaagcaggaggacttccagctgctgtgccccgatggtaccaggaagcctgtgactgaggcccagagctgtcacctagcagcagtccccagtcatgctgtggtctcaaggaaagataaggcggattttgtccgcagaatgctcttcaatcaacaggagctttttggaagaaatgggtttgaatacatgatgttccagctgttcaaatcctcgaccgaggatctgctcttcagcgatgacacagaatgtttggctaaccttcaggacaaaataacttatcaaaaatacttagggccagagtatcttcaggctattgctaacgtgagacaatgcttcccctccgaacttctggatgcctgcactttccatggaaattaa

SEQ ID NO: 3 - DNA, 20 bp, artificial sequence
  chemically syntethized
atgactgttgctaggtcggt

SEQ ID NO: 4 - DNA, 20 bp, artificial sequence
  chemically synthetized
tcgtcaacgtctgtgggctt

SEQ ID NO: 5 - DNA, 20 bp, artificial sequence
  chemically syntethized
tgcgtcttgctgtgcgcggg

SEQ ID NO: 6 - DNA, 20 bp, artificial sequence
  chemically synthetized
tggaaagtgcaggcatccag

SEQ ID NO: 7 - DNA, 726 bp, Isochrysis galbana
  misc_feature (1)..(726): carbonic anhydrase encoding sequence
gatcacaaagtatgctgcaaagcacctccgttcgtcgagcgggctggtcggctcctcggctggcttgcgcccggctggccggctcgcggcggctggtgtgcagcggatacggcagtcttagcctcgcgcgtcacatcaagcacaacgccgactttgtcgagaagaacaaggaccctgtctccgaacatggcgccaagtcgcaccagccgtggtacaggaggatcgggtgcagcgatgcgcgggcctcgctcaacgaattcttcggccagtaccgcggcgaggcctctatgcatcgcaacgtagccaacccggtagtcaacacggacaagaacctcctgtcagtgatgcagtacgtggtcggtgcgctgtgcgtgcccgatatcatcgtgtgcggtcacgacgacagcggcggggtcaaggccacggtgagcaagtcatcgcccgactcgcgcgaccttggctcgccgccggagccgaccaagaagacgacagactccacggtgactgaggaggaggtgcgggctgcacaggctcggtgggcaaactccatcaagaccatcagcagcacctacctcaacggcggcgactacgtccaggccgccgccgacgccgccgacgagctgtacggctacggccgctcgacggtgctcttcaagcccaccatgtgcgccgagtaccagttccgcccgacgggcaacgacgcgatgtcgtacttcgtgggccaca

SEQ ID NO: 8 - DNA, 23 bp, artificial sequence
  chemically synthetized
tacyystacatcggbtgcgtbga

SEQ ID NO: 9 - DNA, 20 bp, artificial sequence
  chemically synthetized
gtggargckrtagacrtcnc

SEQ ID NO: 10 - protein, 8 aa, artificial sequence
  chemically syntehtized
YLYIGCVD

SEQ ID NO: 11 - protein, 7 aa, artificial sequence
  chemically synthetized
RDVYRLH

SEQ ID NO: 12 - DNA, 816 bp, artificial sequence
  chemically synthetized
atgcgcaagctgattgaggggctgcgccacttccggacgtcctattaccccagccaccgcgacctgttcgagcagtttgccaagggccagcatccgcgcgtgctgttcattacgtgcagcgactcccgcatcgatccgaacctgatcacgcagtcgggcatgggggagctgttcgtgatccgcaacgcgggcaacctgatcccgcccttcggcgctgccaacggcggtgagggcgcctccatcgagtacgccatcgcggctctgaacatcgagcacgtggtggtgtgcggccactcgcactgcggtgcgatgaagggcctgctcaagctgaaccaactgcaagaggacatgccgctggtgtacgactggctccagcacgcccaggcgactcgccggctggtgctggacaactacagcggctacgagactgacgacctggtggagatcctggtcgcggagaacgtgctgacccagatcgagaacctcaagacctacccgatcgtccgcagccgcctgttccagggcaagctccagatcttcggctggatctacgaggtggagtcgggcgaggtgctccagattagccggaccagctccgacgacaccggcatcgacgagtgccccgtccgcctgcccgggagccaggagaaggccattctgggccgctgcgtggtgcccctgaccgaggaggtcgccgtggcccctcccgagccggagcccgtgattgccgcggtcgccgctcctcccgcgaactactcctcgcgcggttggctggcgcctgagcagcagcagcgcatctaccggggcaacgccagc

SEQ ID NO: 13 - DNA, 1098 bp, artificial sequence
  chemically synthetized
atggtgcctttttggacgactgtctcccggaacggtagctcggactcggagactaccctccagtccgcgtccaaggcgaccaagcagtacaagtacccgagcctgcgcccttcgcaccgcctgagcctgctgtttctgttccccttccacctgtcggcgaacggcgcctgcttccgctgcacctgcttcagccacttcaagctggagctgcgccgcatggggaacgagagctacgaggacgcgatcgaggcgctgaagaagctgctgatcgagaaggacgatctgaaggatgtggctgccgccaaggtgaagaagatcaccgccgagctgcaagccgcgtccagcagcgactccaagagcttcgaccccgtggagcggatcaaggaggggttcgtgacgttcaagaaggagaagtacgagactaaccctgcgctgtacggcgagctggcgaagggccagtcccccaagtacatggtgttcgcctgctccgacagccgcgtgtgcccgagccacgtcctcgacttccaccccggtgacgctttcgtggtgcgcaacattgccaacatggtgccgcccttcgacaaggtcaagtacgctggcgtgggtgccgctatcgagtacgccgtgctgcatctgaaggtggagaacattgtggtcatcggccactcggcctgcggcggtatcaagggcctgatgtcgtttcccctggacggcaacaacagcaccgacttcatcgaggactgggtgaagatttgcctgcccgccaagtccaaggtcctggcggagtcggagtccagcgcgttcgaggaccagtgcggtcggtgcgagcgcgaggccgtgaacgtgtcgctcgccaacctcctgacgtaccccttcgtgcgcgagggcgtggtgaagggcaccctggccctcaagggcggctattacgacttcgtgaacggcagcttcgagctgtgggagctgcaattcggcatctccccggtccactcgatcaagctttacccgtacgatgtgccggactacgcgggctatccgtacgacgtccccgactacgccggctcgtacccctacgacgtgcccgattacgctgcgcagtaa

SEQ ID NO: 14 - DNA, 1602 bp, artificial sequence
  chemically synthetized
atggtgatccagagcatcacccacctgagccccaacctggccctgcccagccccctgagcgtgtccaccaagaactaccccgtggccgtgatgggcaacatcagcgagcgcgaggagcccaccagcgctaagcgcgtggcagtggtgggcgctggcgtgagcggcctggctgctgcctacaagctgaagtcccacggcctgagcgtgaccctgttcgaggccgacagccgcgccggcggcaagctgaagaccgtgaagaaggacggcttcatctgggacgagggcgccaacaccatgaccgagagcgaggccgaggtgtccagcctgatcgacgacctgggcctgcgcgagaagcagcagctgcccatcagccagaacaagcgctacatcgcccgcgacggcctgcccgtgctgctgcctagcaaccccgccgccctgctgacctccaacatcctgagcgccaagagcaagctgcagatcatgctggagccgttcctgtggcgcaagcacaacgccaccgagctgagcgacgagcacgtgcaggagagcgtgggcgagttcttcgagcgccacttcggcaaggagttcgtggactacgtgatcgaccccttcgtggccggcacctgcggcgacccccagagcctgagcatgcaccacaccttccccgaggtgtggaacatcgagaagcgcttcggcagcgtgttcgccggcctgatccagtccaccctgctgagcaagaaggagaagggcggcgagaacgccagcatcaagaagccccgcgtgcgcggcagcttcagcttccagggcggcatgcagaccctggtggacaccatgtgcaagcagctgggcgaggacgagctgaagctgcagtgcgaggtgctgagcctgagctacaaccagaagggcatccccagcctgggcaactggtccgtgagcagcatgagcaacaacaccagcgaggaccagagctacgacgccgtggtggtgaccgcccccatccgcaacgtgaaggagatgaagatcatgaagttcggcaaccccttcagcctggacttcatccccgaggtgacctacgtgcccctgtccgtgatgatcaccgccttcaagaaggacaaggtgaagcgccccctggagggcttcggcgtgctgatccccagcaaggagcagcacaacggcctgaagaccctgggcaccctgttcagcagcatgatgttccccgaccgcgcccccagcgacatgtgcctgttcaccaccttcgtgggcggctcccgcaaccgcaagctggccaacgccagcaccgacgagctgaagcagatcgtgtccagcgacctgcagcagctgctgggcaccgaggacgagcccagcttcgtgaaccacctgttctggtccaacgccttccccctgtacggccacaactacgactgcgtgctgcgcgccatcgacaagatggagaaggacctgcccggcttcttctacgccggcaaccacaagggcggcctgtccgtgggcaaggccatggccagcggctgcaaggcggccgagctggtgatcagctacctggacagccacatctacgtgaagatggacgagaagaccgcctaa